(19) United States
(12) Patent Application Publication
(10) Pub. No.: US 2026/0130782 A1
(43) Pub. Date: May 14, 2026

(54) ORTHOSIS

(71) Applicant: B6 Products, LLC, Grand Rapids, MI (US)

(72) Inventor: Gabriel B. Beversluis, Grand Rapids, MI (US)

(73) Assignee: B6 Products, LLC, Grand Rapids, MI (US)

(21) Appl. No.: 19/442,231

(22) Filed: Jan. 7, 2026

Related U.S. Application Data
(63) Continuation-in-part of application No. 18/522,395, filed on Nov. 29, 2023, now Pat. No. 12,544,248.
(60) Provisional application No. 63/430,024, filed on Dec. 4, 2022.

(51) Int. Cl. A61F 5/01

(52) U.S. Cl. CPC A61F 5/0127

(57) ABSTRACT

An orthosis comprises a first portion configured for placement on a first portion of a limb and a second portion configured for placement on a second portion of the limb. The first portion and the second portion are attached to one another by at least one joint permitting relative rotation therebetween. A strap is coupled between the first portion and the second portion, at least a portion of the strap being elastic. The strap includes a ratchet mechanism configured to permit incremental adjustment of tension applied to the elastic portion and to selectively release all tension. A first end of the strap is connected to the first portion at a location offset from the at least one joint, and a second end of the strap is connected to the second portion at a location offset from the at least one joint.

### CROSS-REFERENCE TO RELATED APPLICATIONS

The present application is a continuation-in-part of U.S. patent application Ser. No. 18/522,395, filed Nov. 29, 2023, which claims the benefit of U.S. Provisional Application No. 63/430,024, filed Dec. 4, 2022. The entire disclosures of the foregoing applications are hereby incorporated by reference in their entireties.

### TECHNICAL FIELD

The invention generally relates to orthoses and therapeutic procedures using orthoses. Additionally, the invention relates to improved components and systems for use with jointed orthoses configured for positioning, supporting, or applying therapeutic forces to a limb.

### BACKGROUND

Orthoses are used to support, position, align, or apply therapeutic forces to portions of a patient's body, including limbs and joints, to improve function, alignment, or range of motion. A patient's joint alignment or range of motion may have lost range of motion and become misaligned due to conditions such as cerebral palsy, injury, or other similar types of medical disorders.

With particular regard to children, if the joint misalignment or loss of range of motion is not corrected, this condition will likely lead to a permanent condition or deformity as the child ages.

Current orthoses used to improve range of motion and correct such misalignment are expensive to purchase, heavy, bulky, and lack durability. They require tools to operate and are prone to breakage. Therefore, it is desirable to provide an improved orthosis.

In particular, currently available orthoses and orthosis components used for bracing, including the ankle foot orthosis (“AFO”) from Ultraflex Systems and Multimotion from Allard USA, Inc., are: 1) expensive to purchase and may not be covered by insurance because of cost; 2) heavy and bulky and not ideal for pediatric applications; 3) lacking in durability and prone to breakage; and 4) requiring tools to adjust. Therefore, it is desirable to provide for lower-cost orthoses and orthosis components. Additionally, it is desirable to provide an orthosis that is light in weight and may be used for pediatric patients. Finally, it is desirable to have an orthosis with improved durability.

Other current common stretching orthoses, including stretching ankle foot orthoses, use “stretching straps” which extend from one portion of the limb to another to create a stretch. For example, a product manufactured by Cascade Dafo Inc. is the Cascade #9 Stretching AFO; however, this product does not allow movement of the joint while pushing against the resilience of the strap. This can lead to poor compliance and lack of improvement in range of motion. Additionally, such stretching straps do not provide the progression into the improved joint position while in use. Additionally, stretching strap orthoses do not provide a numerical indicator to track progress and limb position. Therefore, providing for an orthosis that permits dynamic stretching, is adjustable, and provides a numerical indicator for the patient is desirable.

Other orthoses such as “Rooster Boot” are known to require tools to adjust and release tension. Such additional tools complicate usage and adoption by a patient. The disclosure of U.S. Pat. No. 9,398,970 is hereby incorporated by reference herein. Moreover, such orthoses may require two separate components, a first component anterior to the ankle to provide stretch, and a second component behind the ankle to “lock” the tension at a fixed position. Therefore, it is desirable to provide a simplified orthosis having a straightforward operation.

### SUMMARY OF THE INVENTION

In an aspect, an orthosis comprises a first portion configured for placement on a first portion of a limb and a second portion configured for placement on a second portion of the limb. The first portion and the second portion are attached to one another by at least one joint permitting relative rotation between the first portion and the second portion. The orthosis includes a strap coupled between the first portion and the second portion, at least a portion of the strap being elastic. The strap has a ratchet mechanism configured to permit incremental adjustment of tension applied to the elastic portion and to selectively release all tension. A first end of the strap is connected to the first portion at a location offset from the at least one joint, and a second end of the strap is connected to the second portion at a location offset from the at least one joint.

In certain embodiments, the orthosis comprises an ankle foot orthosis (“AFO”) that comprises an upper portion configured for placement on a lower leg. The AFO has a lower portion configured for placement on a foot, the upper portion and lower portion being attached to one another by a lateral joint and a medial joint. The lateral joint and medial joint allow rotation of the upper portion with respect to the lower portion. The AFO has a strap, at least a portion of the strap being an elastic portion. The strap has a ratchet mechanism allowing an incremental increase in stretching the elastic portion of the strap. The ratchet mechanism is releasable to release all tension. A first end of the strap is connected to the upper portion offset from and anterior to the lateral joint and the medial joint, and a second end of the strap is connected to the lower portion offset from and anterior to the lateral joint and the medial joint.

In certain embodiments, the strap has a first strap that is inelastic and a second strap that is elastic. The upper portion can have a shell and an inner support structure. The upper portion can have a proximal flare.

In certain embodiments, the upper portion has a strap extending from a first side of the upper portion to a second side of the upper portion. The upper portion, in embodiments, has a support structure having a vertical portion. The vertical portion can comprise a metal bar. The support structure has, in embodiments, a horizontal portion extending proximally.

In embodiments, the support structure has a lower end attached to the lower portion. The lower end of the support structure can have a first bar and a second bar, the first bar may be disposed at an angle with respect to the second bar. For example, the lower end of the support structure can be generally Y-shaped.

The upper portion has an anterior projection that is, in embodiments, offset from a longitudinal axis aligned with the lateral joint and the medial joint. In embodiments, the lateral joint can limit rotation of the upper portion with respect to the lower portion to twenty degrees in a forward direction and twenty degrees in a rearward direction. In embodiments, the limit on rotation in one or both directions is adjustable.

In embodiments, the ratchet mechanism is a ratchet buckle having a button for releasing all tension when the button is pushed. The ratchet buckle can have a rotatable handle. The handle can be a rotatable knob. The upper portion, in embodiments, has an anterior projection, the first end of the strap being connected to the anterior projection.

In embodiments, the ankle foot orthosis has an inner boot. In embodiments, at least one of the lateral joint and the medial joint has a goniometer.

In certain embodiments, the orthosis comprises a knee-ankle-foot orthosis (“KAFO”). The KAFO includes a first portion configured for placement on an upper leg and a second portion configured for placement on a lower leg and foot. The first and second portions are coupled by at least one knee joint permitting relative rotation therebetween, and in certain embodiments further include at least one ankle joint permitting relative rotation between the lower leg and foot. A stretching strap is coupled between the first portion and the second portion at locations offset from the at least one knee joint, the stretching strap including an elastic portion and a ratchet mechanism configured to permit incremental adjustment of tension and selective release of all tension to provide adjustable and progressive stretching across the knee and/or ankle joints.

In certain embodiments, the orthosis comprises a knee orthosis (“KO”). The knee orthosis includes a first portion configured for placement on an upper leg and a second portion configured for placement on a lower leg. The first and second portions are coupled by at least one knee joint permitting relative rotation therebetween. A stretching strap is coupled between the first portion and the second portion at locations offset from the knee joint, the stretching strap including an elastic portion and a ratchet mechanism configured to permit incremental adjustment of tension and selective release of all tension.

In certain embodiments, the orthosis comprises an elbow orthosis (“EO”). The elbow orthosis includes a first portion configured for placement on an upper arm and a second portion configured for placement on a forearm. The first and second portions are coupled by at least one elbow joint permitting flexion and extension. A stretching strap is coupled between the first portion and the second portion at locations offset from the elbow joint, the stretching strap including an elastic portion and a ratchet mechanism configured to permit incremental adjustment of tension and selective release of all tension.

In certain embodiments, the orthosis comprises a hip positioning and extension assist orthosis. The hip orthosis includes a first portion configured for placement on a torso or pelvic region and a second portion configured for placement on at least one thigh. The first and second portions are coupled by at least one hip joint permitting relative rotation. A stretching strap is coupled between the first portion and the second portion at locations offset from the hip joint, the stretching strap including an elastic portion and a ratchet mechanism configured to permit incremental adjustment of tension and selective release of all tension to provide adjustable hip extension assistance.

In certain embodiments, the orthosis comprises an ankle foot orthosis that comprises an upper portion configured for placement on a lower leg, the upper portion having an anterior projection. The orthosis can have a lower portion configured for placement on a foot. The upper portion can have a support structure with a lower end connected to the lower portion with a first bar and a second bar; the first bar being disposed at an angle with respect to the second bar. The upper portion and lower portion can be attached to one another by a lateral joint and a medial joint, the lateral joint and medial joint allowing rotation of the upper portion with respect to the lower portion. The orthosis has a strap offset from the lateral joint and medial joint. The strap includes a first strap, a second strap, at least a portion of at least one of the first strap and second strap being elastic, and a ratchet mechanism connecting the first strap to the second strap. The ratchet mechanism permits an incremental increase in stretching the elastic of the at least a portion of at least one of the first strap and second strap through movement of a handle. The ratchet mechanism is releasable to release all tension. A first end of the first strap is connected to the anterior projection of the upper portion, a second end of the first strap extends through the ratchet mechanism, a first end of the second strap extends through the ratchet mechanism, and a second end of the second strap is connected to the lower portion.

In embodiments, the first strap is inelastic and the second strap is elastic. In embodiments, the upper portion has a shell and an inner support structure. The upper portion may have a proximal flare. In embodiments, the upper portion has a strap extending from a first side of the upper portion to a second side of the upper portion.

The support structure can have a vertical portion. The vertical portion, for example, comprises a metal bar. The support structure can have a horizontal portion extending proximally. In embodiments, the lower end of the support structure is generally Y-shaped.

The anterior projection, in embodiments, is offset from a longitudinal axis aligned with the lateral joint and the medial joint.

In certain embodiments, the lateral joint limits rotation of the upper portion with respect to the lower portion to twenty degrees in a forward direction and twenty degrees in a rearward direction.

The orthosis can further comprise an inner interface structure, such as an inner boot. In embodiments, at least one of the lateral joint and the medial joint has a goniometer.

In a further aspect, a stretching strap for an orthosis is disclosed. The stretching strap includes a first strap, and a second strap; at least a portion of at least one of the first strap and second strap is elastic. The strap has a ratchet mechanism connecting the first strap to the second strap. The ratchet mechanism permits an incremental increase in stretching the elastic portion. The ratchet mechanism is releasable to release all tension. A first end of the first strap is arranged for connection to a portion of an orthosis, a second end of the first strap extends through the ratchet mechanism, a first end of the second strap extends through the ratchet mechanism, and a second end of the second strap is arranged for connection to another portion of the orthosis. In certain embodiments, the ratchet mechanism is a buckle having a button for releasing all tension when the button is pushed. In certain embodiments, the first strap is inelastic and the second strap is elastic. In certain embodiments, the ratchet mechanism has a rotatable handle. In embodiments, the handle is a rotatable knob.

In an aspect, a stretching strap for an orthosis has a first strap, a second strap, at least a portion of at least one of the first strap and second strap is elastic, and a ratchet mechanism connecting the first strap to the second strap. The ratchet mechanism permits an incremental increase in stretching the elastic portion and is releasable to release all tension. A first end of the first strap is arranged for connection to a portion of an orthosis, a second end of the first strap extends through the ratchet mechanism, a first end of the second strap extends through the ratchet mechanism, and a second end of the second strap is arranged for connection to another portion of the orthosis. In certain embodiments, the ratchet mechanism is a buckle having a button for releasing all tension when the button is pushed, or when the button is pulled in other embodiments. In certain embodiments, the first strap is inelastic and the second strap is elastic. In certain embodiments, the ratchet mechanism has a handle. The handle may be a rotatable handle. In embodiments, the handle is a rotatable knob. In embodiments, the rotatable knob is pushed or pulled to release all tension.

An orthosis and/or the stretching strap disclosed herein have many advantages. An orthosis that provides for parents and juvenile patients to adjust as needed to either 1) increase the tension for greater stretching; or 2) to decrease the tension such as, for example, where musculature is contracted and there is a desire to improve patient comfort.

An orthosis can be provided with a numeric scale to monitor the current stretching tension setting and to serve as a reference point for changes in tension and/or position.

An orthosis can be provided with an easy method to “unlock” the tension strap in order to remove all tension from the patient's affected limb. With the strap unlocked, the orthosis can be properly applied to the patient. A patient's contracted limb is more easily placed into the orthosis with the strap unlocked.

The orthosis, in embodiments, has a dynamic function which allows the patient to move against the resilience of the strap and actively extend their limb when they feel the need to stretch. The dynamic function also provides progressive movement to continually improve the position of the limb during use, without requiring adjustment.

The orthosis has an adjustable function to allow for a slight incremental movement of the patient's limb. The patient's limb moves into improved angles for range of motion, improving during each session of use. Over time, as the patient's range of motion improves, the strap will continue to push the patient into an improved position. The combination of dynamic and adjustable tension allows for the orthosis to be used on patients with even more severe limitations on joint movement. The orthosis provides continual stretching therapy across at least 40 degrees of angular movement, from the position of 20 degrees in plantar flexion to 20 degrees in dorsiflexion.

A stretching strap is disclosed herein, which may be used with an orthosis. The stretching strap may also be retrofitted into existing AFOs, knee braces, elbow braces, hip braces, et cetera. The stretching strap may include an elastomeric band, in embodiments. In embodiments, the stretching strap includes an elastomeric band connected to a rigid band, and having a ratcheting buckle.

Regardless of the application on an AFO or other device, the stretching strap provides: 1) a low cost option to achieve dynamic and adjustable stretching; 2) adjustability without requiring tools; 3) the ability to be retrofitted into multiple types of custom or prefabricated orthoses; 4) a ratcheting feature that allows for increasing tension until an effective stretch is obtained without complicated instructions; 5) a simple push button tension release to remove the tension (once tension is released the ratcheting feature can then be used to apply tension again); 6) a numeric scale for tension adjustment which allows for targeting appropriate stretching and goal setting; 7) a dynamic stretching strap which creates greater improvement in range of motion by gently pushing against the patients movement; 8) a stretching band that allows patients to move and extend their joints actively against tension, helping with specificity, patient discomfort, and the sensation of feeling trapped; 9) a lighter, less bulky device which is less likely to get tangled in sheets at night, less likely to bang on doors, furniture, or walls, and less likely to cause damage or breakage; 10) a device with easily replaceable parts; and 11) a goniometer for measuring the range of motion.

This summary is neither intended nor should be construed as being representative of the full extent and scope of the present disclosure. The present disclosure is set forth in various levels of detail in the summary as well as in the attached drawings and detailed description, and no limitation as to the scope of the present disclosure is intended by the inclusion of elements, or the exclusion of elements. Additional aspects of the present disclosure will become more readily apparent from the detailed description, particularly when taken together with the drawings.

The above-described benefits, embodiments, and or characterizations are not necessarily complete or exhaustive. Other benefits, embodiments, and/or characterizations of the present disclosure are possible.

### BRIEF DESCRIPTION OF THE DRAWINGS

The accompanying drawings, which are incorporated in and constitute a part of the present disclosure, illustrate preferred embodiments of the invention and, together with the description, serve to explain the present disclosure. Embodiments of the invention are in no way limited by the following figures:

FIG. 1 is a lateral side elevation view of an ankle foot orthosis according to an embodiment, shown in a 90 degree or right angle orientation;

FIG. 2 is a medial side elevation view of the ankle foot orthosis of FIG. 1;

FIG. 3 is a front elevation view of the ankle foot orthosis of FIGS. 1 and 2;

FIG. 4 is a rear elevation view of the ankle foot orthosis of FIGS. 1 through 3;

FIG. 5 is a lateral side elevation view of the ankle foot orthosis of FIGS. 1 through 4 shown in a dorsiflexion position of 20 degrees;

FIG. 6 is a medial side elevation view of the ankle foot orthosis of FIGS. 1 through 5 shown in a dorsiflexion position of 20 degrees;

FIG. 7 is a lateral side elevation view of the ankle foot orthosis of FIGS. 1 through 6 shown in a plantar flexion position of 20 degrees;

FIG. 8 is a front elevation view of a stretching strap according to an embodiment;

FIG. 9 is a rear elevation view of the stretching strap of FIG. 8;

FIG. 10 is a perspective view of an ankle foot orthosis showing an inner liner removed from a lower portion of the ankle foot orthosis;

FIG. 11 is a front elevation view of a stretching strap according to a further embodiment;

FIG. 12 is a lateral side elevation view of an ankle foot orthosis according to a further embodiment;

FIG. 13 is a lateral side elevation view of an ankle foot orthosis according to another embodiment;

FIG. 14 is a lateral side elevation view of a knee-ankle-foot orthosis according to an embodiment;

FIG. 15 is an enlarged view of a top portion of the knee-ankle-foot orthosis of FIG. 14;

FIG. 16 is a medial side elevation view of the knee-ankle-foot orthosis of FIGS. 14 and 15;

FIG. 17 is a front elevation view of the knee-ankle-foot of FIGS. 14 through 16;

FIG. 18 is a rear elevation view of the knee-ankle-foot orthosis of FIGS. 14 through 17;

FIG. 19 is a lateral side elevation view of a knee orthosis according to an embodiment;

FIG. 20 is a front elevation view of the knee orthosis of FIG. 19;

FIG. 21 is a lateral side elevation view of an elbow orthosis according to an embodiment;

FIG. 22 is a front elevation view of the elbow orthosis of FIG. 21;

FIG. 23 is a rear elevation view of the elbow orthosis of FIGS. 21 and 22;

FIG. 24 is a front elevation view of the hip positioning and extension assist orthosis according to an embodiment; and

FIG. 25 is a side elevation view of the hip positioning and extension assist orthosis of FIG. 24.

### DETAILED DESCRIPTION

An orthosis for allowing a patient to engage in therapeutic stretching is disclosed herein. Additionally, improved components for use with an orthosis are disclosed.

As used herein, the term “orthosis” refers to an externally applied device configured to support, align, prevent, correct, assist, resist, or otherwise influence the position, movement, or function of one or more anatomical structures or joints of a patient.

An orthosis may span one or more joints, may be rigid, semi-rigid, or flexible, and may provide static, dynamic, resistive, or assistive forces. Unless otherwise indicated, the term “orthosis” as used herein includes, without limitation, ankle-foot orthoses (AFO), knee-ankle-foot orthoses (KAFO), knee orthoses (KO), elbow orthoses (EO), hip orthoses, hip-knee orthoses (HKO), hip-knee-ankle-foot orthoses (HKAFO), reciprocating gait orthoses (RGO), and combinations or variations thereof, including devices incorporating adjustable or dynamic stretching components, wherein the abbreviations used herein are provided for convenience only and are not intended to limit the scope of the disclosure.

### Ankle Foot Orthosis (“AFO”)

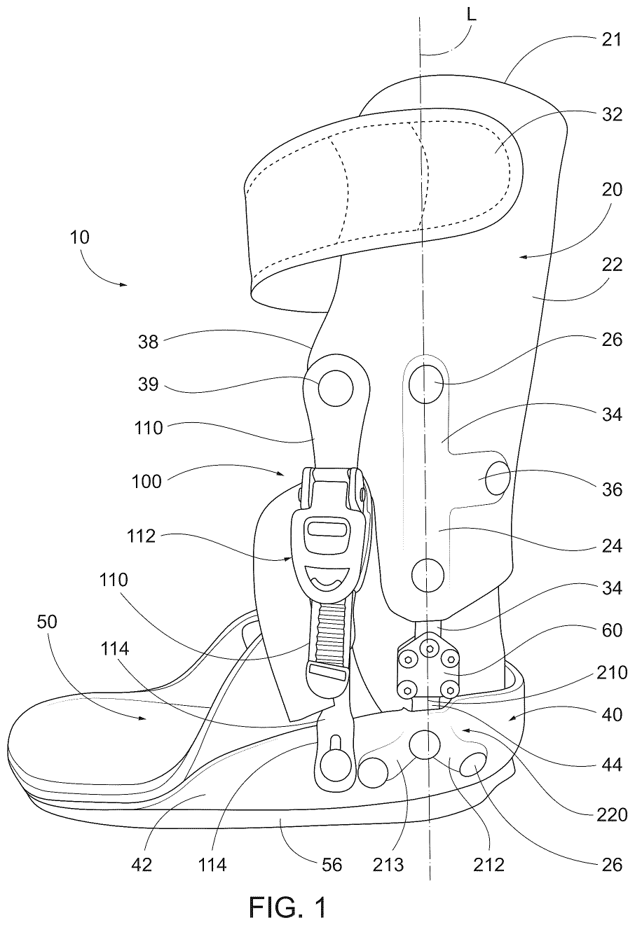

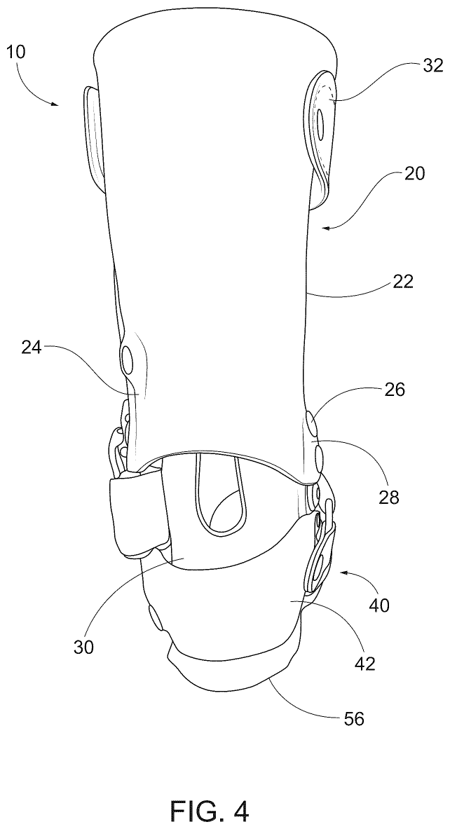

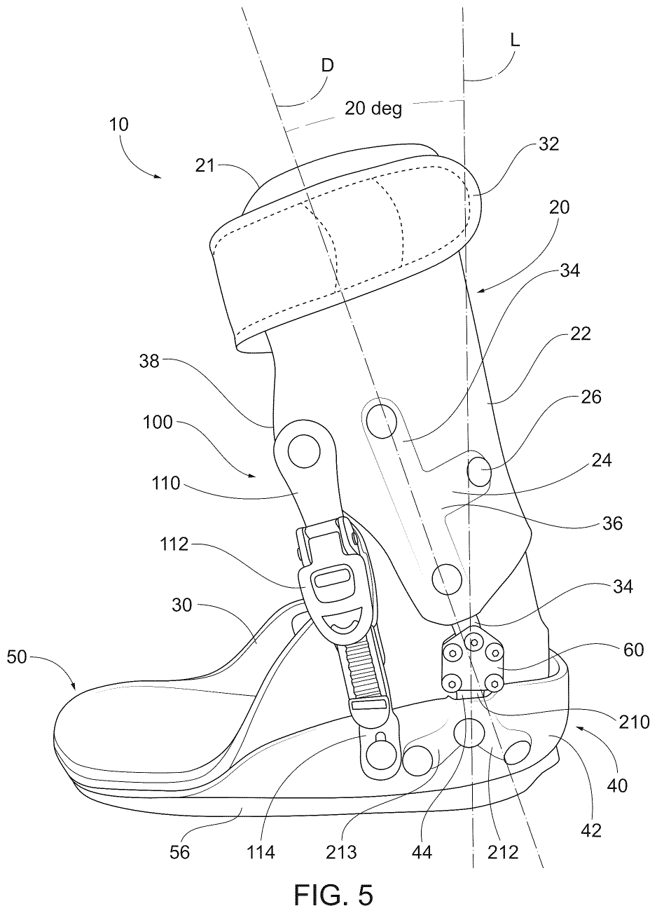

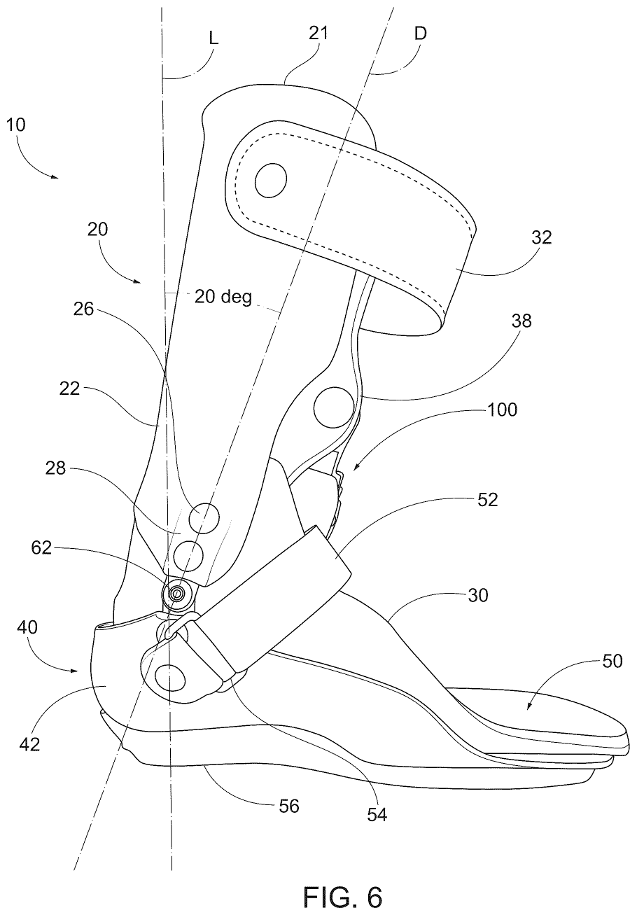

Reference is made to FIGS. 1 through 7 which illustrate an ankle foot orthosis 10 including a stretching strap 100 also referred to as Dynamic Adjustable Stretching Strap (DASS). In certain embodiments, the stretching strap 100 provides resistance against movement of the foot. In certain embodiments, the stretching strap is adjustable to provide different range of motion and/or different tension. In certain embodiments, the stretching strap 100 has a portion or portions that are elastic and which may provide greater tension as the strap 100 is stretched.

The ankle foot orthosis 10 has an upper or calf portion 20 and a lower or foot portion 40. Both portions include a shell 22, 42, respectively, which can be formed of rigid plastic or molded plastic.

The upper portion 20 has an outer support structure 24 and an inner support structure 28 attached to the shell 22 by connectors 26. A proximally extending flair 21 is provided on the upper portion 20, in embodiments. The orthosis 10, in certain embodiments, has an inner liner 30 made of flexible material such as a pliable foam to comfortably extend around the patient's calf. Extending from one side of the upper portion 20 to the other side is an upper strap or proximal strap 32. The strap 32 connects both sides once in place upon a patient. The proximal strap 32 may be affixed using hook and loop fasteners, snaps, buckles, or other means.

The outer support structure 24 has a vertical portion 34 extending along the calf and a horizontal portion 36 connected to the vertical portion 34 and extending rearwardly. The vertical portion 34 extends along a longitudinal axis “L”. The vertical portion 34 and horizontal portion 36 comprise, in certain embodiments, a metal bar or bars.

The vertical portion 34 and horizontal portion 36 comprise, in certain embodiments a metal bar or bars attached to the outer support structure 24. The vertical portion 34 and horizontal portion 36 can be attached using rivets, screws, or other connectors. In certain embodiments, the horizontal portion 36 is omitted. In embodiments, a vertical portion and horizontal portion is formed from a thicker area or areas of plastic in the outer support structure 24. As shown in FIG. 1, the lateral hinge or joint 60 connects an upper portion of the vertical portion 44 to a lower portion of the vertical portion 34.

The vertical portion 34 can be connected to an outer support structure 44 that can have a first bar 210 and a second bar 212, the first bar being disposed at an angle with respect to the second bar. The second bar may extend toward a heel of the orthosis. The lower end 220 can be formed as a Y-shaped pair of bars. For example, a second bar 212 may extend toward the heel of the orthosis, and a third bar 213 can extend toward a toe of the orthosis.

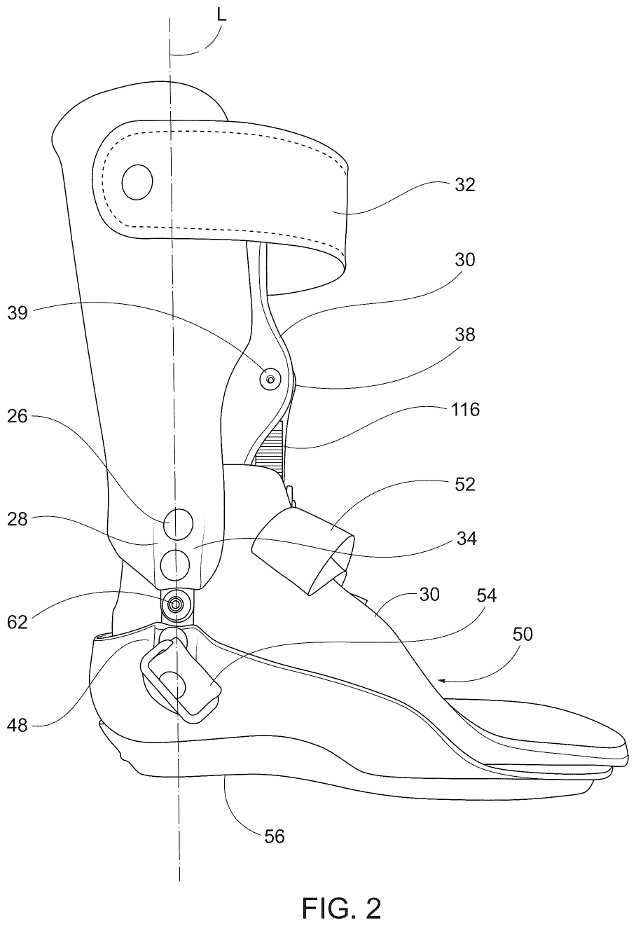

In embodiments, a lateral side of the outer support structure 24 and the medial side of the outer support structure 24 can have a vertical portion 34 and/or horizontal portion 36. In embodiments, the medial side has a shortened vertical portion 34 comprising a metal bar, plastic material, or other supporting structure (see FIG. 2).

The upper portion 20 has an anterior offset or forward member 38 extending at a point generally mid-body. The forward member 38 is offset from the longitudinal axis L. The forward member 38 serves as the point at which stretching strap 100 is attached. Attached at this point, the stretching strap 100 is generally parallel to the longitudinal axis L and anterior to the longitudinal axis L.

The lower portion 40 has an outer support structure 44 and an inner support structure 48 attached to the shell by connectors 26. The lower portion 40 includes a molded inner boot or inner liner 50 made of a flexible material such as a pliable foam that provides pressure distribution. Extending from one side of the foot portion is a lower strap or in-step ankle strap 52 to connect both sides once in place on a patient's foot. The strap 52 may be fixed by being threaded through, for example, a loop 54, then the strap is folded upon itself and secured using hook and loop fasteners, snaps, buckles, or other means. The lower portion 40 has a sole 56 affixed to it with treads for ambulation while avoiding slipping.

The upper portion 20 and lower portion 40, are connected together by their support structures through a plurality of joints. In certain embodiments, there is a lateral hinge or outer joint 60 and a medial hinge or inner joint 62.

In embodiments, the lateral hinge or outer joint 60 is designed to permit rotation for ankle joint rotation with dorsiflexion and or plantar flexion limits. As shown, in certain embodiments, rotation is limited to approximately 20 degrees in one direction and approximately 20 degrees in the other direction. In embodiments, the lateral hinge 60 is limited, whereas the medial hinge 62 does not limit travel in either direction. In embodiments, either or both hinges have a mechanical stop to limit rotation. In certain embodiments, the limit on rotation in one or both directions is adjustable.

The stretching strap 100 has an upper strap or upper band 110, a lower strap or lower band 114 and a ratchet mechanism 112 connecting the upper strap and lower strap, in certain embodiments. In embodiments, the ratchet mechanism is a buckle. In embodiments, the upper strap 110 is made of plastic or other material that is inelastic. In embodiments the lower strap 114 is elastic and can be an elastic urethane band or an elastic strap available from Gaffney Technology LLC. The upper strap is attached to the offset 38 by a connector 39. Attaching the stretching strap 100 at the offset 38 maintains more consistent mechanical advantage through dorsiflexion and plantar flexion.

The ratchet buckle 112, in embodiments, is like those used for ski and snowboard bindings and is designed to be tightened but not loosened. In other words, the operation of the buckle allows motion of one strap with respect to the other in one direction only. The strap can be released by pushing the buckle 112, allowing the upper strap 110 to travel within the buckle 112 and releasing tension.

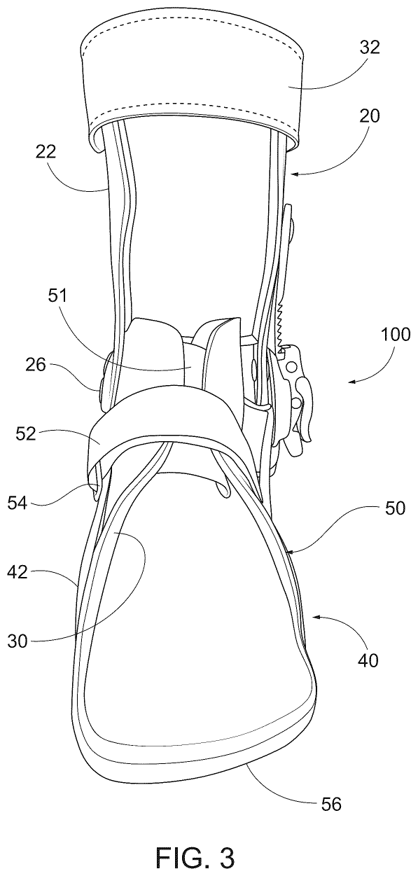

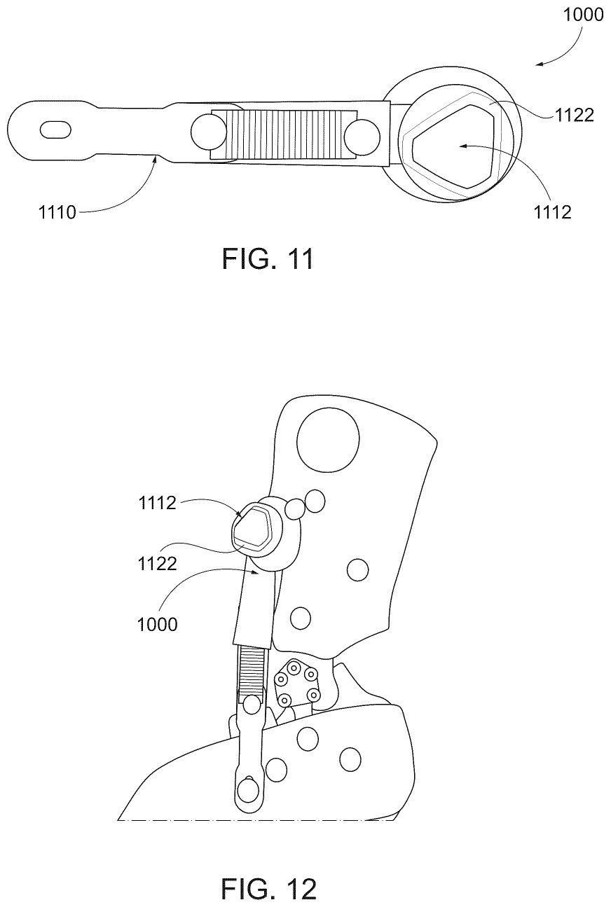

In embodiments, as shown in FIGS. 3 and 11, the inner boot 50 is shown as a wrap around boot with dorsal pads 51.

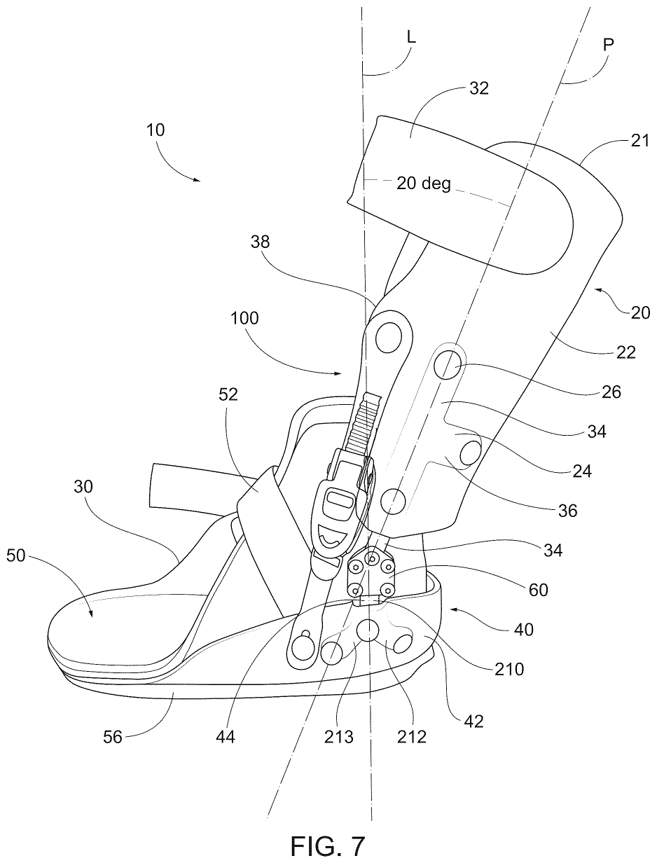

As seen in FIG. 7, the upper portion 20 can also rotate in either direction relative to the lower portion 40 about hinges 60, 62. The vertical portion 34 is positioned along axis “P” at 20 degrees to the longitudinal axis L. In embodiments, the hinge or hinges may freely rotate when the stretching strap 100 is not in place. The stretching strap 100 adds variable resistance in plantar flexion. The stretching strap 100 assists in movement of the ankle in dorsiflexion.

In use, the ankle foot orthosis 10 is placed upon a patient's foot and adjusted for therapy. The stretching strap AFO is designed to provide progressive and adjustable stretching to contracted musculature exhibiting range of most motion loss in the gastrocnemius and soleus muscles. Range of motion loss in this muscle group is very common in patients with a variety of diagnoses including cerebral palsy, spina bifida, muscular dystrophy, autism, idiopathic toe walking, club foot, spinal muscular atrophy, as well as many other neuromuscular and orthopedic diagnoses. The stretching strap AFO is designed to provide essential characteristics which will provide for improved compliance by patients, improved ease of donning the AFO and use, improved adjustability as needed depending on patient progress, and for improved protection of fragile bone and joint structures while isolating the stretching forces to the Achilles tendon and calf musculature.

In order to properly put on the AFO, it is very important that the patient or patient's assistant is able to position the patient's foot and lower leg into the AFO with the heel seated properly and the bones and joints held in a corrected neutral position. The stretching AFO allows for push button tension release to remove all tension from the AFO in order to facilitate this process.

When a patient's musculature is contracted with range of motion loss, it can be very difficult to properly seat the patient's foot and lower leg into an AFO. Once the patient's limb is properly seated and secured into the AFO, then the incremental tension adjustment may be applied.

Using the numeric scale which corresponds to incremental adjustments on the strap, the stretching tension will be applied up to the desired amount, taking into account the patient's comfort level. This incremental adjustment with the numeric scale allows for a continual reference point to ensure that the appropriate amount of stretch is being applied. The incremental tension adjustment also allows for an easily understandable program to increase the stretching tension over time, achieve maximum results, and maximize range of motion improvement.

The dynamic and progressive tension applied by the combination of the ratcheting component and elastomeric band also provides additional benefits. The patient will have a greater sense of freedom of movement of the affected limb and is able to actively push against the elastic band to stretch or to deal with muscle contractions occurring. The elastomeric band provides progressive stretch tension which allows for improved stretching capability even as the patient's range of motion changes during use. The elastic portion or portions elastically increase tension as they stretch. This capability of providing an adjustable and progressive stretching mechanism allows for greater patient comfort and greater patient compliance as well as greater potential range of motion improvements.

Additionally, the stretching AFO features, in certain embodiments, a dorsiflexion limit (which may consist of a Sure Step DA joint 60) or other joint with motion limiting capabilities. The limit may be set at a full 20 degrees of dorsiflexion, representing a full normal range of motion for the ankle joint, or any desired angle depending on the patient's needs. In embodiments, a hinge joint with a mechanical stop is used.

The dorsiflexion limit on the AFO ensures that the patient's ankle joint will not be stretched beyond the anatomical limitations, preventing potential injury. The dorsi flexion limit also provides a tangible “finish line” for the stretching therapy when the range of motion improves. The dorsiflexion limit helps patients and caregivers visualize the range of motion improvement being made as well as the need for further progress.

The AFO, in embodiments, has an internal inner boot which wraps around the foot and ankle. In embodiments, the inner boot is pliable and is molded to the foot. In embodiments, the inner boot is arranged to maintain the proper position of the foot and ankle in a neutral position. This can prevent stretching forces from inadvertently damaging the fragile joint structure of the subtalar joints and midtarsal joints. The molded inner boot also serves to distribute pressure created by stretching forces and the instep strap in order to prevent pressure sores and improve patient comfort and compliance.

### Stretching Strap

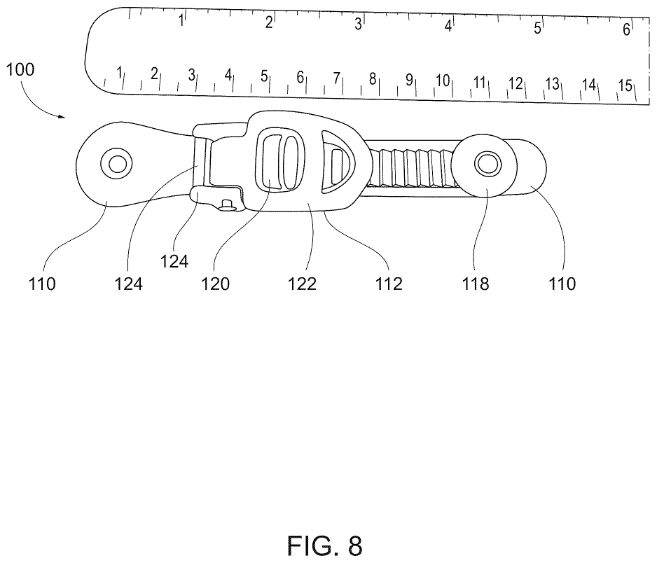

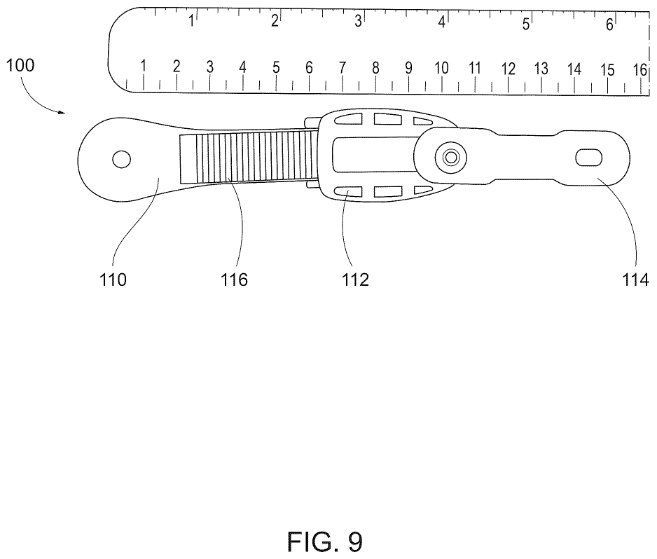

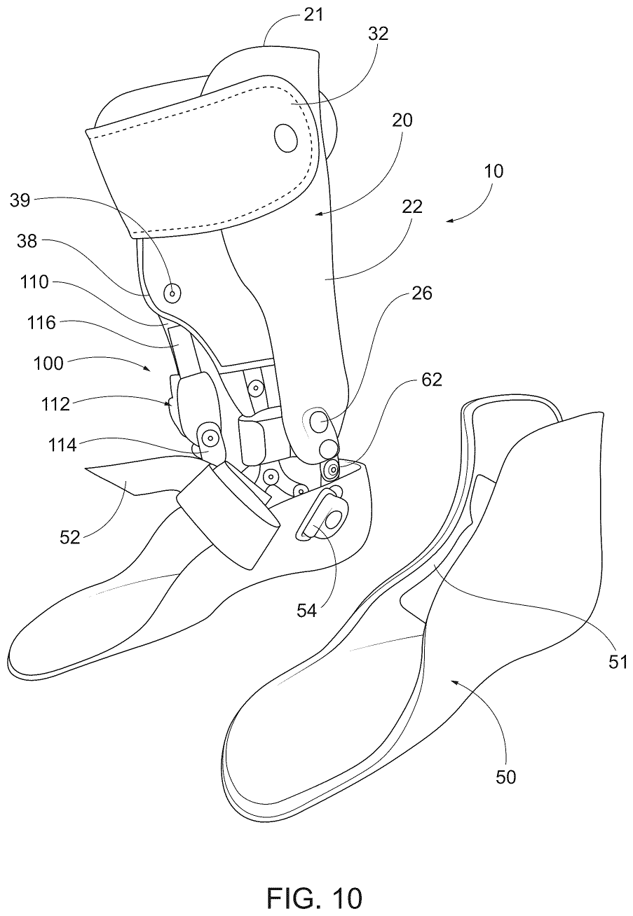

As seen in FIGS. 8 and 9, the stretching strap 100, in embodiments, has an upper strap 110, a lower strap 114, and a ratcheting buckle 112 connecting the upper strap 110 and the lower strap 114. The lower strap 114 is made of plastic that is elastic. In certain embodiments, the elastic strap is a manufactured elastic urethane band or band available from Gaffney Technology, LLC. The upper strap 110 is designed to be inelastic. Indicia 116 are provided on the upper strap 110, in embodiments. A stopper 118 is provided in embodiments to prevent the strap 110 from sliding through the ratcheting buckle 112. The ratchet buckle 112 has, in embodiments, a handle 122 that can be rotated about a hinge 124 to increase tension and, in embodiments, the buckle 112 has a button 120 that can be pushed to release all tension.

### Adjustable Knob

In certain embodiments the orthosis has a ratchet mechanism in the form of an adjustable knob. The stretching strap 1000, in embodiments, has a strap member 1110, and a ratcheting knob 1112. The strap member 1110 is made of plastic that is elastic. In embodiments, indicia are provided on the strap member. The ratchet knob 1112 has, in embodiments, a handle 1122 that can be rotated to increase tension.

To begin increasing the tension on the stretching strap 1000, knob 1112 is pressed down. The knob is rotatable in a clockwise or counterclockwise direction to increase tension on the stretching strap 1000. The knob has a similar ratchet mechanism as ratchet buckle 112 to allow movement of the strap in one direction only. To release tension, the knob 1112 is pulled upwardly. See FIGS. 11 and 12. An example of such a knob is the L6 dial, which is used on the La Sportiva BOA running shoe.

### Goniometer

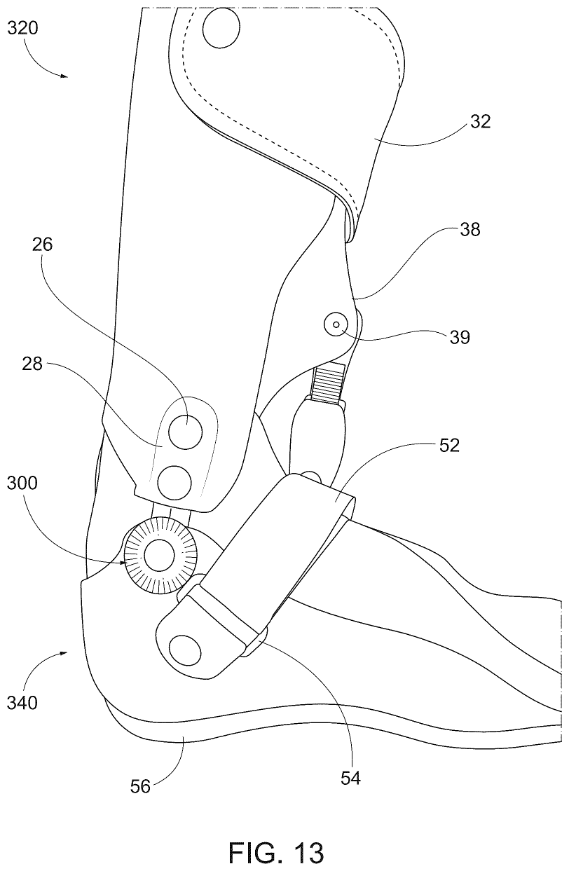

In embodiments, a goniometer 300 to measure angular joint position and determine how much progress is being made. As seen in FIG. 13 for the AFO, the goniometer 300 is formed on the lateral hinge, in certain embodiments. The goniometer measures the angular position of the upper portion 320 relative to the lower portion 340 before the patient moves their foot, pressing against the resistance of the stretching strap. As the foot is moved, the degree of movement is shown on the goniometer 300.

The goniometer measures actual angular positioning and range of motion progress being made with the AFO, while the numeric scale on the stretching strap measures the amount of force being applied to the contracted musculature. This combination of measurement capability and incremental adjustment, give patients and medical staff the ability to track progress, and make informed decisions about whether or not the stretching strap tension is needed to be increased.

### Pronounced Projections

The embodiment shown in FIG. 13 also has an anterior projection that is more pronounced, and extends farther in the anterior direction, than the anterior projection 38 shown in FIG. 1. This keeps the stretching strap components much more streamlined to the patient's leg. This allows for the use of a knee immobilizer orthosis, to be worn in conjunction with the AFO without disrupting or changing the stretching capability, or interfering with the stretching strap. In addition, all, or almost all, of the numeric scale on the stretching strap is visible.

The forces exerted with the stretching strap AFO adjustment are very inconsistent, depending on the angle of the foot to the leg. The forces exerted by the stretching strap varies, depending on the distance of the strap anterior to the ankle joint axis. When the foot is plantar flexed, the stretching strap design moves significantly closer to the ankle axis, greatly diminishing the stretching force. When the foot is dorsiflexed, the stretching strap moves much further away from the ankle joint axis, creating a much stronger stretching force.

The location of the stretching strap provides approximately a ‘parallel pull’ which has a much more constant stretching force though all angles of movement of the ankle.

The anterior attachment projection shown in FIG. 13 locates the stretching strap on the anterior lateral portion of the AFO which avoids the strap getting tangled up with the opposite foot, avoids getting tangled in sheets, and minimizes bulk.

### Knee-Ankle-Foot Orthosis (“KAFO”)

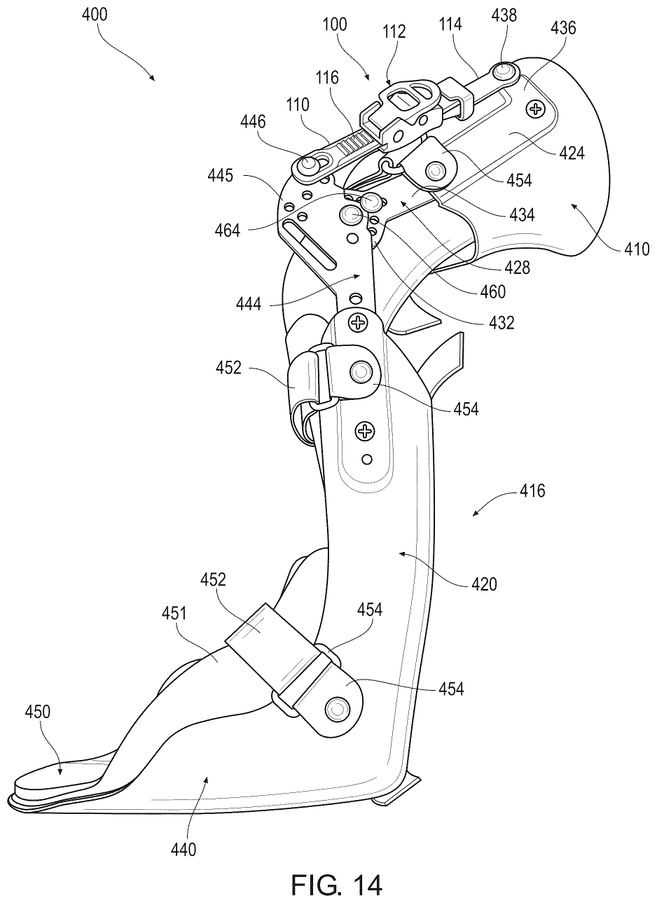

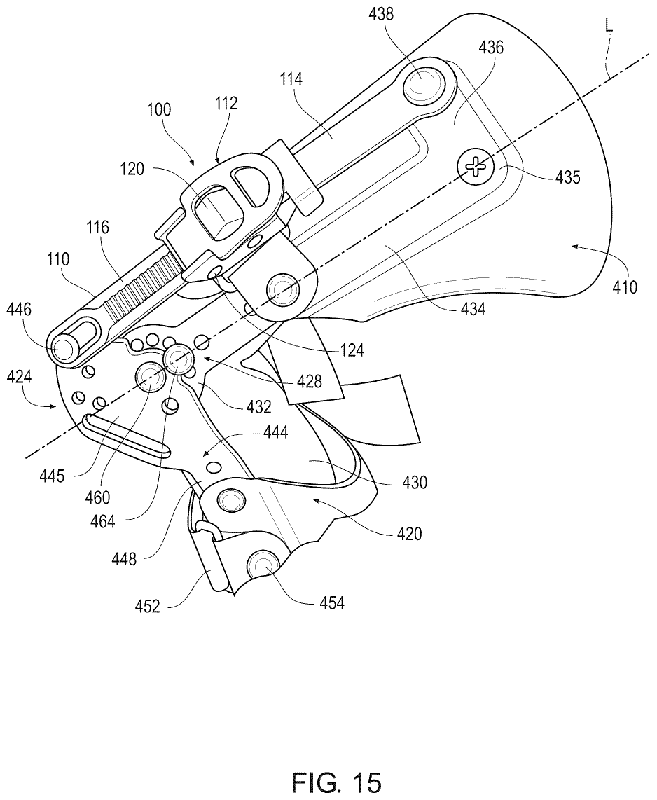

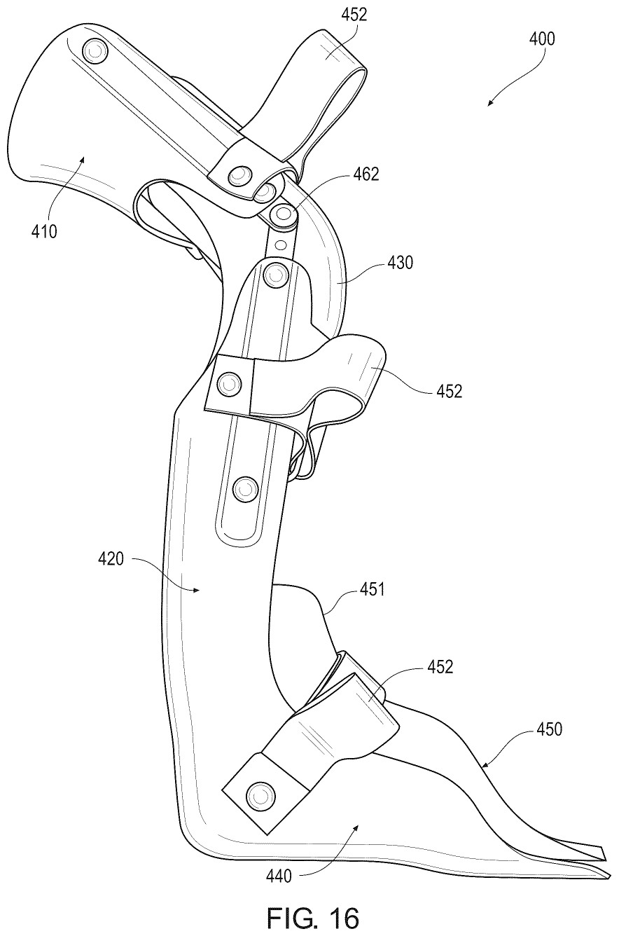

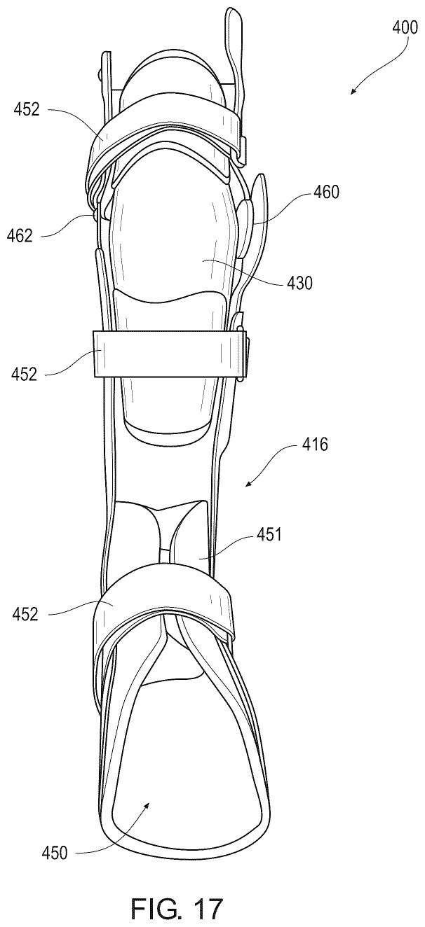

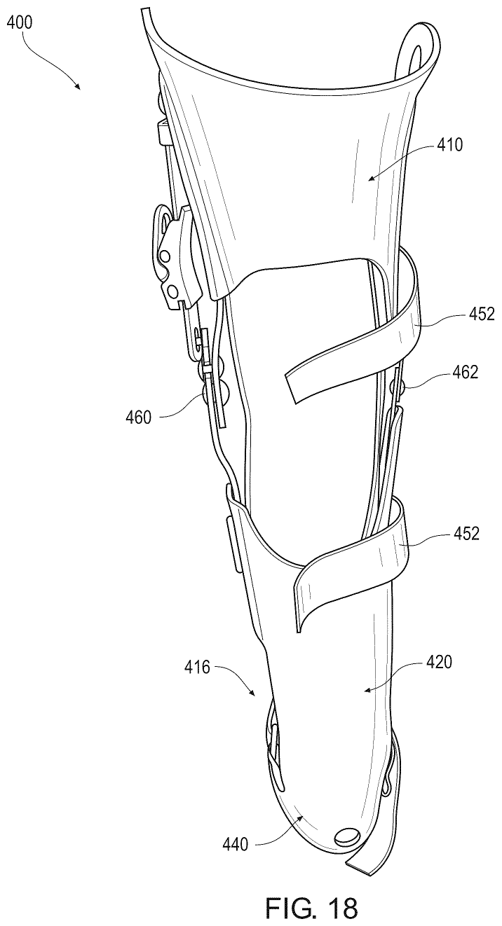

Reference is made to FIGS. 14 through 18 which illustrate a knee-ankle-foot orthosis (“KAFO”) 400 including a stretching strap 100. The stretching strap 100 provides resistance against movement of the knee. As previously disclosed, the stretching strap is adjustable to provide different range of motion and/or different tension. In certain embodiments, the stretching strap 100 has a portion or portions that are elastic and which may provide greater tension as the strap 100 is stretched.

The KAFO includes an upper or thigh portion 410 configured for placement on an upper leg, and a lower portion 416 formed by the calf portion 420 and the foot portion 440. The thigh portion 410 and the lower portion 416 collectively define first and second portions of the orthosis. In the embodiment shown, the calf portion 420 and foot portion 440 are formed together as a single structure with no separate ankle articulation; however, in alternative embodiments the calf portion 420 and the foot portion 440 are connected by a lateral ankle joint and a medial ankle joint, which allow rotation analogous to the ankle joints using a DASS or stretching strap 100 as disclosed earlier in the ankle foot orthosis.

The orthosis 400, in certain embodiments, has an inner liner 430 made of flexible material such as a pliable foam to comfortably extend around the patient's thigh and an inner boot 450 with dorsal pads 451 made of a similar material. Extending from one side of the orthosis 400 to the other is a strap 452 used to connect both sides of the orthosis together once the patient's knee, ankle, and foot are in the orthosis. The strap 452 may be fixed by being threaded through, for example, a loop 454, then the strap is folded upon itself and secured using hook and loop fasteners, snaps, buckles, or other means. The foot portion may have a sole affixed to it with treads for ambulation while avoiding slipping.

The thigh portion 410 and the calf portion 420 are connected by a lateral knee joint 460 and a medial knee joint 462 (collectively, the “knee joint assembly”), permitting knee flexion and extension with optional motion-limiting stop 464. As shown, in certain embodiments, rotation is limited to approximately 20 degrees in one direction and approximately 20 degrees in the other direction. In this embodiment, the lateral hinge 460 has adjustable limits into flexion and extension depending on patient need, whereas the medial hinge 462 does not limit travel in either direction.

The stretching strap 100 has an upper strap or upper band 110, a lower strap or lower band 114 and a ratchet mechanism 112 operatively connecting the upper strap 100 and lower strap 114, in certain embodiments. In embodiments, the ratchet mechanism 112 comprises a buckle configured to incrementally tension the lower strap 114 relative to the upper strap 110. The upper strap 110 may be formed from a substantially inelastic material, such as a rigid or semi-rigid plastic. The lower strap 114 may be formed from an elastic material including elastic urethane bands or an elastic strap available from Gaffney Technology, LLC, allowing controlled elongation during tensioning.

An adjustable jointed arm assembly 424 is provided having an upper arm mounting plate 428 and a lower arm mounting plate 444. The upper arm mounting plate 428 has an upper region 435, a lower region 432, and a longitudinal portion 434 defining axis “L.” The longitudinal portion 434 includes a series of apertures arranged along its length to position fasteners, and a fastening point may be provided at the upper region to secure the thigh portion 410.

Extending perpendicular from the upper region 435 is a horizontal portion 436 that terminates at a forward mounting point 438. The forward mounting point 438 provides an attachment location for the stretching strap assembly 100, the attachment location being offset from the knee joint assembly, and in certain embodiments, elastic strap 114 is secured to the forward mounting point 438 by a connector.

The lower region 432 is formed as a substantially circular plate positioned at the lower end of the longitudinal portion 434. The circular plate includes a central pivot aperture and a plurality of circumferentially spaced apertures arranged around the perimeter of the circular plate.

These circumferential apertures are configured to receive a pivot fastener in alignment with corresponding apertures of the lower arm mounting plate 444, thereby enabling selectable angular indexing of the lower arm mounting plate relative to the upper arm mounting plate. Because the lower region 432 is a full circle, the fastener may be received at any of the distributed apertures around the circumference, providing a wide range of discrete pivot positions and allowing precise adjustment of the relative angle between the plates.

The lower arm mounting plate 444 of the adjustable joint arm assembly 424 includes an arcuate pivot plate 445 defining a curved sector of a circle. The arcuate plate 445 includes a central pivot aperture located near its inner radius and a series of circumferentially spaced apertures arranged along its outer curved perimeter. These circumferential apertures are configured to selectively align with corresponding apertures in the lower region 432 of the upper arm mounting plate 428, thereby permitting indexed angular adjustment between the two components.

Along the outer circumference of the arcuate plate 445, an attachment feature is provided for securing a portion of the stretching strap assembly 100. The stretching strap may be coupled through one of the circumferential apertures or through a dedicated connector mounted adjacent the curved edge of the arcuate plate, enabling the strap to apply tension at a location offset from the knee joint assembly in a direction generally parallel to the longitudinal axis of the support structure.

Extending downward from the arcuate pivot plate 445 is a longitudinal extension 448 that projects into and along the user's calf region. This longitudinal extension 448 is an elongated structural member that permits the lower arm mounting plate 444 to be fastened to the user's calf by means of straps or cuffs and functions to transmit forces from the calf to the pivot plate during adjustment or movement.

The upper arm mounting plate 428 and a lower arm mounting plate 444 are joined at a connection point or lateral knee joint 460, which forms the rotational interface between the circular lower region 432 of the upper arm mounting plate and the arcuate pivot plate of the lower arm mounting plate 444. The connection point 460 includes a pivot fastener received through the central pivot apertures of both plates, permitting relative rotation about a shared pivot axis. In certain embodiments, a rotation stop 464 is provided adjacent the connection point 460 to limit the angular displacement of the lower arm mounting plate 444 relative to the upper arm mounting plate 428, thereby preventing rotation beyond a predefined range to ensure safe and controlled operation. Additionally, the stretching strap assembly 100 is connected to the lower arm mounting plate 444 at an attachment point 446 positioned along the outer circumference of the arcuate plate, enabling the stretching strap to apply tension directly to the lower portion during adjustment or use.

In use, the orthosis 400 is placed upon the patient's leg and adjusted to achieve the desired therapeutic stretch. The stretching-strap KAFO 400 is designed to provide progressive and adjustable stretching to contracted musculature exhibiting loss of range of motion in the hamstrings, gastrocnemius, soleus, and other muscle groups that cross the knee and ankle joints. Loss of extension at the knee and dorsiflexion at the ankle is common in patients with a wide range of neuromuscular and orthopedic conditions, including cerebral palsy, spina bifida, muscular dystrophy, autism, idiopathic toe walking, clubfoot, spinal muscular atrophy, stroke, traumatic injury, and prolonged immobilization.

The stretching-strap KAFO 400 provides essential therapeutic characteristics that improve patient compliance, enhance ease of donning and use, and enable fine adjustability as treatment progresses. The ratcheting stretching strap allows controlled, incremental increases in tension while protecting fragile bone and joint structures of the knee, ankle, and foot. The orthosis is designed to isolate the stretching forces to the hamstrings, gastrocnemius-soleus complex, and associated musculotendinous structures while maintaining proper alignment of the femur, tibia, and foot. This targeted application of force maximizes therapeutic benefit, reduces secondary stresses on adjacent joints, and supports safe, effective restoration of functional range of motion.

### Knee Orthosis (“KO”)

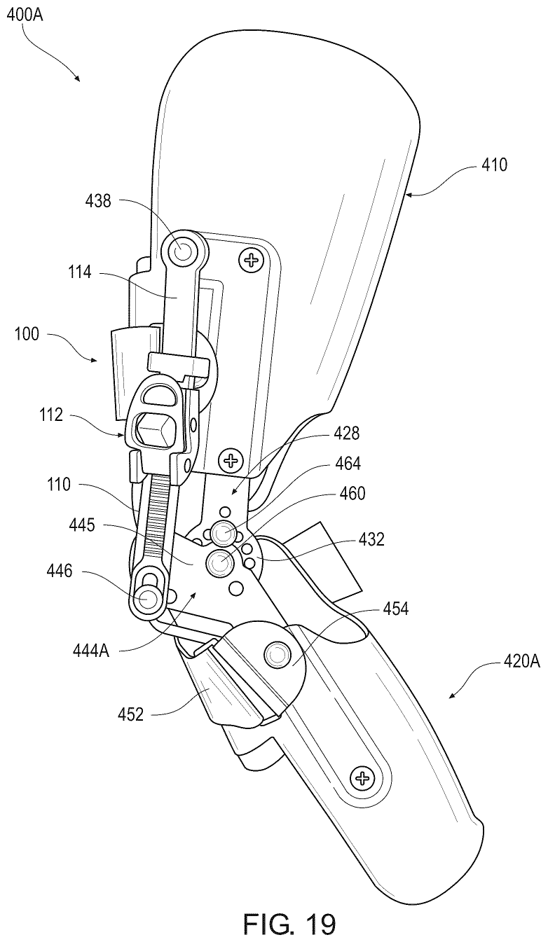

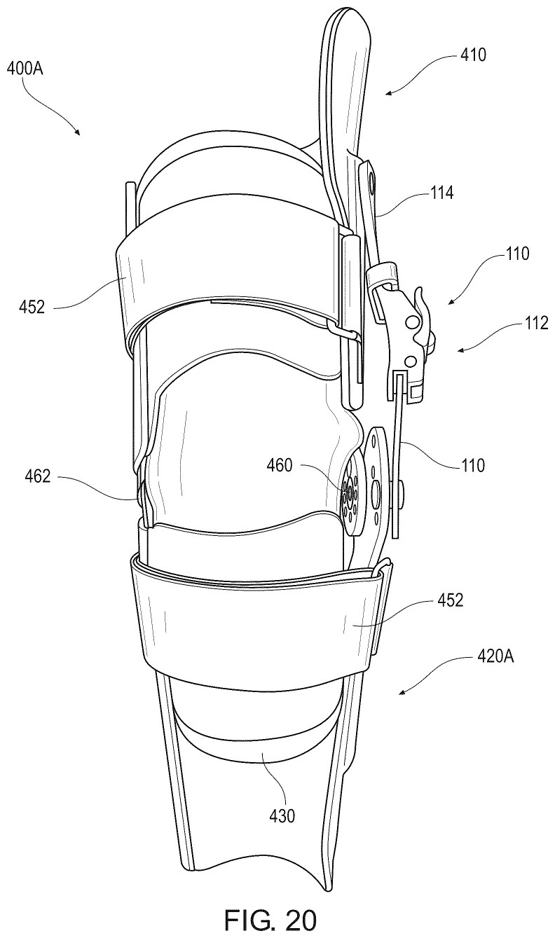

Reference is made to FIGS. 19 and 20 illustrating a knee orthosis (“KO”) 400A constructed using the same structural components, numbering, and general architecture described above for the knee-ankle-foot orthosis (“KAFO”) 400, with the exception that the KO 400A omits the distal ankle and foot portions. As with the KAFO, the KO includes a stretching strap assembly 100 configured to apply adjustable resistance and controlled tension across the knee joint to facilitate therapeutic stretching or positioning. The stretching strap assembly 100 in the KO functions identically to that previously described, and may include an upper strap 110, lower strap 114, and ratchet mechanism 112 for incremental tensioning.

The KO includes an upper or thigh portion 410 configured for placement on the patient's upper leg, and a lower or calf portion 420A configured for securement around the patient's lower leg below the knee. The thigh portion 410 and calf portion 420A collectively define first and second portions of the orthosis. Unlike the KAFO, which includes a foot portion 440 and optionally ankle joints for distal articulation, the KO terminates at the calf region and does not include the additional ankle-foot structural components. The calf portion 420A may include an inner liner 430 or similar padding to comfortably engage the patient's lower leg, and a strap 452 or other fastening mechanism may extend across the orthosis to secure the thigh and calf portions around the patient's limb.

The thigh portion 410 and calf portion 420A are connected by a lateral knee joint 460 and a medial knee joint 462, collectively forming the knee joint assembly. In certain embodiments, these joints may be constructed identically to the joint assemblies described for the KAFO, including a pivot fastener, rotational connection point, and an optional motion-limiting stop 464 configured to limit flexion or extension to a predetermined angular range. In certain embodiments, one of the joints (e.g., the lateral joint 460) includes a mechanical stop 464 providing limited rotation, whereas the opposing joint (e.g., medial joint 462) may permit free rotation without restriction.

In the KO configuration, the stretching strap assembly 100 is operably connected between the upper arm mounting plate 428 secured to the thigh portion 410 and the lower arm mounting plate 444A secured to the calf portion 420A. The stretching strap attachment points, including the upper mounting point 438 and lower attachment point 446, may be identical to those used in the KAFO structure. The stretching strap applies controlled, progressive tension to the musculature crossing the knee joint—without imparting forces beyond the calf, as no foot or ankle assemblies are present in the KO.

The KO utilizes the same adjustable jointed arm assembly 424 described for the KAFO, including the upper arm mounting plate 428, lower arm mounting plate 444, circular plate 432, arcuate plate 445, pivot point 460, circumferential apertures, and rotation-limiting stop 464. In the KO, these components function identically to allow adjustable angular indexing and secure locking of the knee angle. The longitudinal extension 448 of the lower arm mounting plate 444 similarly extends along the calf region to provide stable attachment and force transmission, but does not extend distally into any ankle-foot structure.

In use, the knee orthosis (“KO”) 400A is placed upon the patient's leg and adjusted to provide the desired therapeutic stretch across the knee joint. The KO employs the same stretching strap assembly 100, jointed adjustment assembly 424, and structural components described for the KAFO 400, except that the KO omits the distal ankle and foot portions. The KO is designed to provide progressive and adjustable stretching to contracted musculature that crosses the knee joint, including the hamstrings, quadriceps, and other soft-tissue structures responsible for knee flexion and extension. Loss of knee range of motion, such as reduced knee extension or flexion, is common in patients with a variety of neuromuscular and orthopedic diagnoses, including cerebral palsy, stroke, muscular dystrophy, post-surgical stiffness, traumatic injury, and prolonged immobilization.

Because the KO terminates at the calf and does not include ankle or foot components, the orthosis isolates stretching forces exclusively to the knee joint and associated musculotendinous units without applying distal tensile loads to the ankle complex. The ratcheting stretching strap allows controlled, incremental increases in tension, permitting the clinician or patient to gradually lengthen contracted tissues while protecting fragile bone and joint structures of the knee. As with the KAFO, the KO is designed to improve patient compliance and allow fine adjustability throughout treatment. The targeted application of force across the knee maximizes therapeutic benefit, minimizes stress on adjacent joints, and supports safe, effective restoration of functional range of motion.

### Elbow Orthosis (“EO”)

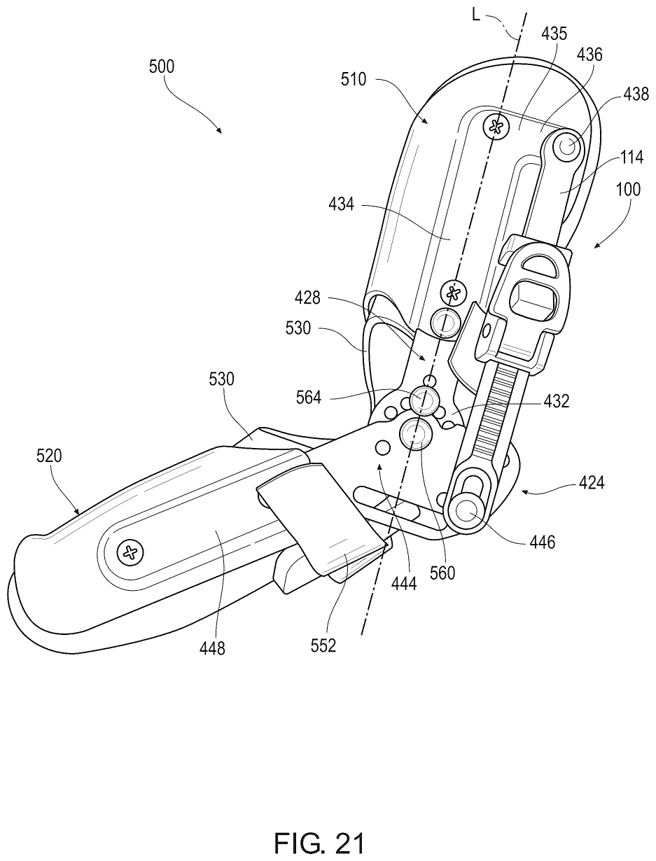

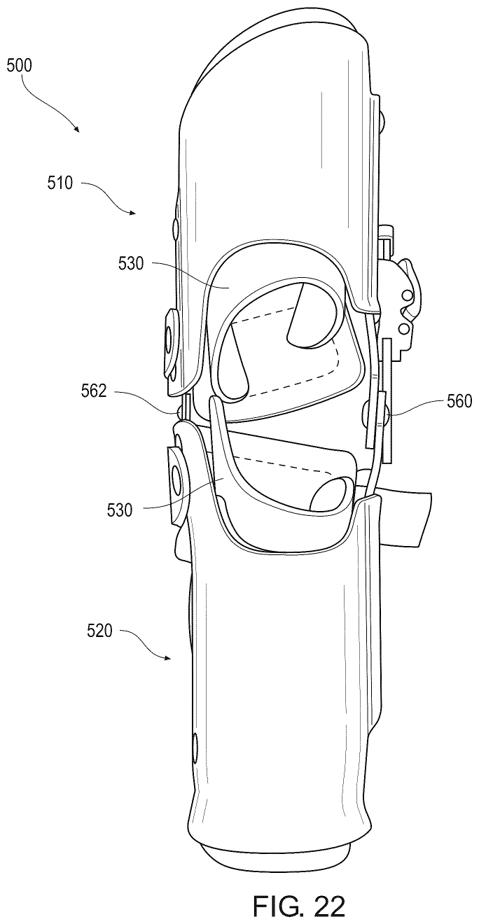

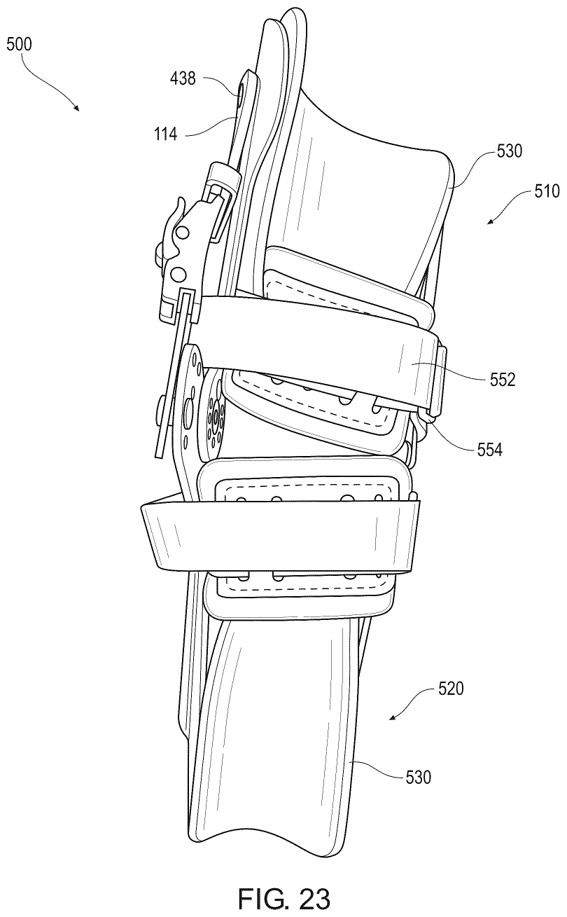

Reference is made to FIGS. 21 through 23 which illustrate an elbow orthosis (“EO”) 500 including a stretching strap 100. The stretching strap 100 provides resistance against flexion or extension of the elbow joint. As previously disclosed, the stretching strap 100 is adjustable to provide different ranges of motion and/or different levels of tension. In certain embodiments, the stretching strap 100 has a portion or portions that are elastic, which may apply progressively increasing tension as the strap 100 is stretched.

The EO 500 includes an upper arm portion 510 configured for placement around the patient's upper arm, and a forearm portion 520 configured for placement around the patient's forearm. The upper arm portion 510 and forearm portion 520 collectively define first and second portions of the orthosis. In certain embodiments, the upper arm portion 510 and forearm portion 520 are thermoformed shells contoured to the user's anatomy and secured by straps, cuffs, or pads. These portions may be lined with padding or an inner liner 530 made of flexible material such as pliable foam to provide comfort and uniform pressure distribution along the arm.

Extending from one side of the orthosis 500 to the other may be one or more straps 552 used to secure the upper arm portion 510 and forearm portion 520 around the patient's limb. The strap 552 may be threaded through a loop or anchor 554 and then folded back upon itself and secured using hook and loop fasteners, buckles, snaps, or equivalent structures.

The upper arm portion 510 and forearm portion 520 are connected by a lateral elbow joint 560 and a medial elbow joint 562 (collectively, the “elbow joint assembly”). In certain embodiments, these joints permit controlled elbow flexion and extension. In certain embodiments, one or both of the elbow joints 560, 562 include a motion-limiting stop 564 to restrict rotation beyond a selected angular range. As in the other orthosis disclosed herein, one joint (e.g., lateral 560) may incorporate angular limits while the opposing joint (e.g., medial 562) allows unrestricted movement.

An adjustable jointed arm assembly 424 is provided having an upper arm mounting plate 428 and a lower arm mounting plate 444. The upper arm mounting plate 428 has an upper region 435, a lower region 432, and a longitudinal portion 434 defining axis “L.” The longitudinal portion 434 includes a series of apertures arranged along its length to position fasteners, and a fastening point may be provided at the upper region 435 to secure the upper arm portion 510.

Extending perpendicular from the upper region 435 is a horizontal portion 436 that terminates at a mounting point 438. The mounting point 438 provides an attachment location for the stretching strap assembly 100, the attachment location being offset from the elbow joint, and in embodiments, elastic strap 114 is secured to the mounting point 438 by a connector.

The lower region 432 is formed as a substantially circular plate positioned at the lower end of the longitudinal portion 434. The circular plate includes a central pivot aperture and a plurality of circumferentially spaced apertures arranged around the perimeter of the plate.

These circumferential apertures are configured to receive a pivot fastener in alignment with corresponding apertures of the lower arm mounting plate 444, thereby enabling selectable angular indexing of the lower arm mounting plate relative to the upper arm mounting plate. Because the lower region 432 is a full circle, the fastener may be received at any of the distributed apertures around the circumference, providing a wide range of discrete pivot positions and allowing precise adjustment of the relative angle between the plates.

The lower arm mounting plate 444 of the adjustable joint arm assembly 424 includes an arcuate pivot plate 445 defining a curved sector of a circle. The arcuate plate 445 includes a central pivot aperture located near its inner radius and a series of circumferentially spaced apertures arranged along its outer curved perimeter. These circumferential apertures are configured to selectively align with corresponding apertures in the lower region 432 of the upper arm mounting plate 428, thereby permitting indexed angular adjustment between the two components.

Along the outer circumference of the arcuate plate 445, an attachment feature 446 is provided for securing a portion of the stretching strap assembly 100. The stretching strap 100 may be coupled through one of the circumferential apertures or through a dedicated connector mounted adjacent the curved edge of the arcuate plate, enabling the strap to apply tension at a location offset from the elbow joint in a direction generally parallel to the longitudinal axis of the support structure.

Extending downward from the arcuate pivot plate 445 is a longitudinal extension 448 that projects into and along the user's forearm region. This longitudinal extension 448 is an elongated structural member that permits the lower arm mounting plate 444 to be fastened to the user's forearm by means of straps or cuffs and functions to transmit forces from the forearm to the pivot plate during adjustment or movement.

The upper arm mounting plate 428 and lower arm mounting plate 444 are joined at a connection point forming an elbow joint 560, which forms the rotational interface between the circular lower region 432 of the upper arm mounting plate and the arcuate pivot plate of the lower arm mounting plate. The connection point 560 includes a pivot fastener received through the central pivot apertures of both plates, permitting relative rotation about a shared pivot axis. In certain embodiments, a rotation stop 564 is provided adjacent the connection point 560 to limit the angular displacement of the lower arm mounting plate 444 relative to the upper arm mounting plate 428, thereby preventing rotation beyond a predefined range to ensure safe and controlled operation. Additionally, the stretching strap assembly 100 is connected to the lower arm mounting plate 444 at an attachment point 446 positioned along the outer circumference of the arcuate plate 445, enabling the stretching strap to apply tension directly to the lower arm mounting plate at a location offset from the elbow joint during adjustment or use.

In use, the elbow orthosis 500 is placed upon the patient's arm and adjusted to achieve the desired therapeutic stretch across the elbow joint. The stretching-strap EO 500 is designed to provide progressive and adjustable stretching to contracted musculature exhibiting loss of range of motion in the biceps, triceps, brachialis, brachioradialis, and other soft-tissue structures that cross the elbow. Loss of elbow extension or flexion is common in patients with a wide range of neuromuscular and orthopedic conditions, including cerebral palsy, brachial plexus injury, muscular dystrophy, spasticity disorders, traumatic injury, stroke, postoperative immobilization, and arthrofibrosis following surgical intervention.

The stretching-strap EO 500 provides essential therapeutic characteristics that improve patient compliance, enhance ease of donning and use, and enable fine adjustability as treatment progresses. The ratcheting stretching strap 100 allows controlled, incremental increases in tension while protecting fragile bone and joint structures of the elbow and surrounding tissues. The orthosis is designed to isolate the stretching forces to the targeted musculotendinous units of the upper arm and forearm while maintaining proper alignment between the humerus and ulna/radius. This targeted application of force maximizes therapeutic benefit, reduces secondary stresses on adjacent shoulder and wrist joints, and supports safe, effective restoration of functional range of motion at the elbow.

### Hip Positioning and Extension Assist (“HIP-PEA”) Orthosis

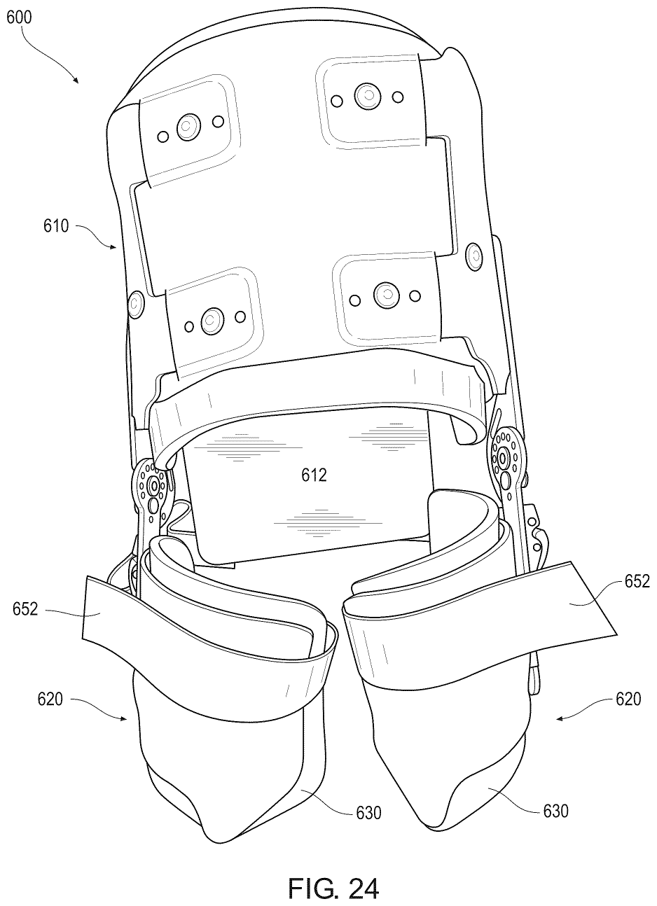

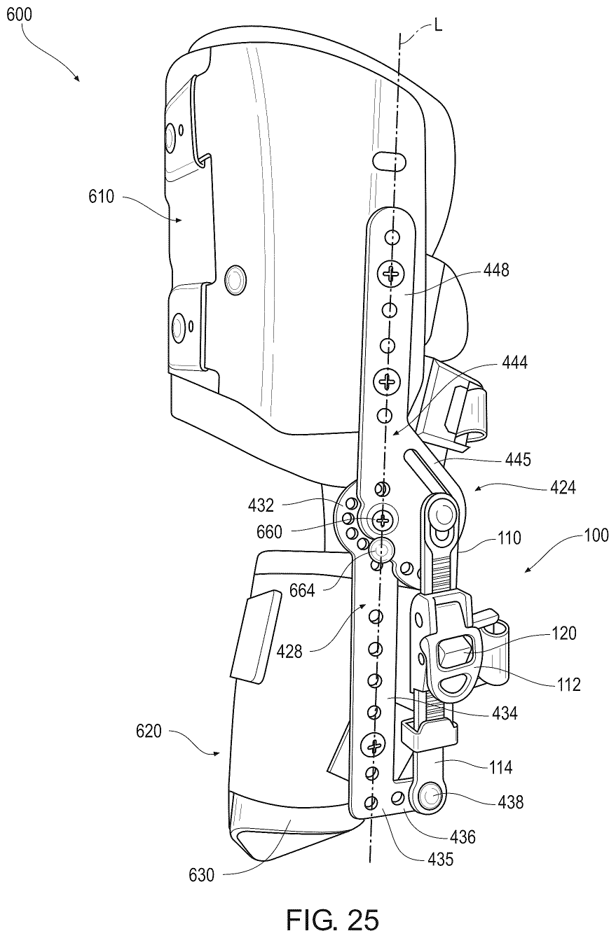

Reference is made to FIGS. 24 and 25 which illustrate a hip positioning and extension assist orthosis (“HIP-PEA”) orthosis 600. The HIP-PEA incorporates the stretching strap 100, which functions as a stretching strap assembly that provides adjustable, progressive resistance and extension assist forces across the hip joint. The HIP-PEA orthosis 600 uses a three-point extension system to apply controlled extension moments across at least one hip joint, and is intended for patients, often children, with hip extensor weakness, hip flexor tightness, spasticity, or hip flexion contractures, such as patients with cerebral palsy and spina bifida.

The HIP-PEA orthosis 600 includes a front of the body or anterior waist portion 610 configured to extend across the torso from approximately the xiphoid process to the anterior superior iliac spines (ASIS). The anterior waist portion 610 serves as the proximal anchor of the three-point extension system and defines a first portion of the orthosis configured for placement on a first portion of a limb or body segment. A posterior gluteal pad 612 is configured to rest beneath the patient's pelvis and press against the buttocks, forming the posterior counterforce needed to generate hip extension.

The orthosis further includes a pair of thigh cuffs 620 configured to extend along the anterior aspects of the patient's thighs. The thigh cuffs 620 serve as the distal anterior contact points of the three-point extension system and collectively define a second portion of the orthosis configured for placement on a second portion of the limb. When extension-assist tension is applied through the stretching strap 100, the thigh cuffs transmit anteriorly directed forces that promote hip extension. For comfort and stability, the anterior waist portion 610, gluteal pad 612, and thigh cuffs 620 may include inner liners 630 composed of soft, pliable cushioning materials. These components are secured around the body using straps 652 and loops 654, which may include hook-and-loop fasteners, buckles, snaps, magnetic connectors, or combinations thereof.

Each thigh cuff 620 is connected to the anterior waist portion 610 by an adjustable joint arm assembly 424. The adjustable joint arm assembly 424 includes an upper mounting plate 428 secured to the corresponding thigh cuff 620 and a lower mounting plate 444 secured to the anterior waist portion 610. The upper mounting plate 428 includes an upper region 435 affixed to the thigh cuff, a longitudinal portion 434 defining axis “L” with a series of apertures for fastener placement, and a lower region 432 formed as a substantially circular plate. The circular lower region 432 includes a central pivot aperture and a plurality of circumferentially spaced apertures arranged around its perimeter.

Extending from the upper region 435 is a horizontal portion 436 terminating at a forward mounting point 438. The forward mounting point 438 serves as an attachment location for the stretching strap 100, the attachment location being offset from the hip joint. In certain embodiments, the elastic strap 114 is secured to the forward mounting point 438 by a connector, allowing extension-assist forces to act through the adjustable joint arm assembly 424.

The lower mounting plate 444 includes an arcuate pivot plate 445 defining a curved sector of a circle. The arcuate pivot plate 445 includes a central pivot aperture positioned near its inner radius and a series of circumferentially spaced apertures arranged along the arcuate outer perimeter. These apertures selectively align with corresponding apertures in the circular lower region 432 of the upper mounting plate 428, permitting indexed angular adjustment between the two components. Extending upward from the arcuate pivot plate 445 to the pelvic region is a longitudinal extension 448 which provides structural support and transmits extension forces from the adjustable joint arm assembly into the anterior waist portion 610.

The upper mounting plate 428 and lower mounting plate 444 are joined at a connection point forming a hip joint 660. A pivot fastener is received through the aligned central pivot apertures of both plates, permitting controlled rotation about a shared pivot axis. In certain embodiments, a rotation stop 664 is positioned adjacent the connection point 660 to limit hip flexion or extension within predetermined bounds, ensuring safe and controlled movement during use. The stretching strap 100 is secured to the lower mounting plate 444 at an attachment point 446 positioned along the arcuate outer perimeter of the arcuate pivot plate 445, enabling the stretching strap 100 to apply tension at a location offset from the hip joint.

The stretching strap 100 includes a ratcheting strap 110, an elastic strap 114, and a ratcheting mechanism 112 operatively connecting the straps. The ratcheting mechanism 112 may include a release button 120 or other release actuator and incremental locking positions that permit the tension applied across the hip joint to be increased one step at a time. In certain embodiments, a numerical tension scale is provided adjacent the stretching strap 100, enabling caregivers and therapists to measure, reproduce, and adjust the extension-assist level provided to the patient.

In use, the HIP-PEA orthosis 600 is placed upon the patient to provide adjustable and progressive assistance into hip extension. The orthosis is typically applied with the patient lying supine, on their back, face upward. The magnetic or quick-disconnect buckles on the anterior waist portion 610 are released, permitting the anterior waist portion 610 to be opened. The anterior waist portion 610 is then positioned across the anterior torso and secured snugly above the pelvis. The caregiver or therapist lifts the patient's pelvis slightly to position the gluteal pad 612 beneath the buttocks. The thigh cuffs 620 are then wrapped around the anterior thighs and secured using straps 652 and associated loops 654, forming a three-point extension system consisting of the anterior waist portion 610, the gluteal pad 612, and the thigh cuffs 620.

Once the HIP-PEA orthosis 600 is properly positioned, the caregiver adjusts the extension-assist tension using the stretching strap 100. The stretching strap 100 includes a ratcheting strap 110 operatively connected to an elastic strap 114 through a ratcheting mechanism 112. Actuation of the release mechanism of the ratcheting mechanism 112 permits free flexion of the hip joint for positioning. Incrementally advancing the ratcheting mechanism 112 increases the hip extension-assist force one step at a time, with the selected tension level visible on the numerical scale adjacent the ratchet. The recommended treatment begins at a relatively low tension level to allow gradual adaptation while the patient continues to activate their own hip extensor musculature.

The HIP-PEA orthosis 600 is designed to allow dynamic movement during therapeutic activities. The patient may continue to flex the hips slightly against the spring-biased resistance of the stretching strap 100, permitting active participation in strengthening while receiving partial assistance toward hip extension. As the patient adapts, the caregiver or therapist may increase the extension-assist tension by one numerical increment at a time over the course of days or weeks, depending on clinical progress. If fatigue occurs readily, additional assistive tension may be provided; conversely, if the patient demonstrates improved independent hip extension, the tension may be reduced accordingly.

The HIP-PEA orthosis 600 may be utilized during standing, stepping, gait training, or other therapeutic activities requiring improved hip posture. The three-point extension system assists the patient in maintaining hip extension and reduces excessive adduction or scissoring by promoting neutral frontal-plane alignment. Because the orthosis imparts an anterior extension moment at the hip from a location offset from the joint, the patient may have limited ability to rapidly flex the hips to sit or cushion a fall. Accordingly, the HIP-PEA 600 is intended for use only under direct adult or therapist supervision, especially during upright or ambulatory activities, similar to the precautions used with hip-knee orthosis (HKO), ankle-foot orthosis (AFO), hip-knee-ankle-foot orthosis (HKAFO), or reciprocating gait orthosis (RGO).

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Other embodiments of the invention will be apparent to those skilled in the art from consideration of the specification and practice of disclosed herein. Although specific advantages have been enumerated, various embodiments may include some, none, or all of the enumerated advantages. It is intended that the embodiments described be considered as exemplary only, with a true scope and spirit of the invention being indicated by the appended claims. Moreover, none of the features disclosed in this specification should be construed as essential elements, and therefore, no disclosed features should be construed as being part of any claim unless expressly recited. In addition, it should be understood that any of the features disclosed in any particular embodiment may be incorporated in whole or in part any of the other embodiments.

In any interpretation of the claims appended hereto, it is noted that no claims or claim elements are intended to invoke or be interpreted under 35 USC 112 (f) unless the words “means for” or “step for” are explicitly used in the particular claim.

All cited references are incorporated herein by reference hereby.

Although embodiments have been disclosed, it is not desired to be limited thereto. While various embodiments of the present disclosure have been described in detail, it is apparent that modifications and alterations of those embodiments will occur to those skilled in the art. However, it is to be expressly understood that such modifications and alterations are within the scope and spirit of the present disclosure.

The foregoing discussion of the disclosure has been presented for purposes of illustration and description. The foregoing is not intended to limit the disclosure to the form or forms disclosed herein. In the foregoing detailed description, for example, various features of the disclosure are grouped together in one or more embodiments. This method of disclosure is not to be interpreted as reflecting an intention that the claims require more features that are expressly recited in the claims. Rather, as the following claims reflect, inventive aspects lie in less than all features of a single embodiment. Thus, the following claims are hereby incorporated into this detailed description, with each claim standing on its own as a separate preferred embodiment of the disclosure. Moreover, though the present disclosure has included description of one or more embodiments and certain variations and modifications, other variations and modifications are within the scope of the disclosure such as, for example, as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to dedicate any patentable subject matter to the public.

What is claimed is:

1. An orthosis comprising:
a first portion configured for placement on a first portion of a limb;
a second portion configured for placement on a second portion of a limb;
the first portion and the second portion attached to one another by at least one joint permitting relative rotation between the first portion and the second portion; and,
a strap, at least a portion of the strap being an elastic portion, the strap having a ratchet mechanism allowing an incremental increase in stretching the elastic portion of the strap and being releasable to release all tension, a first end of the strap connected to the first portion at a location offset from the at least one joint, and a second end of the strap connected to the second portion at a location offset from the at least one joint.

2. The orthosis of claim 1, wherein the strap has a first strap that is inelastic and a second strap that is elastic.

3. The orthosis of claim 1, wherein the first portion has a shell and an inner support structure.

4. The orthosis of claim 1, wherein the first portion has a proximal flare.

5. The orthosis of claim 1, wherein the first portion comprises a strap extending from a first side of the first portion to a second side of the first portion.

6. The orthosis of claim 1, wherein the first portion comprises a support structure having a vertical portion.

7. The orthosis of claim 6, wherein the vertical portion comprises a metal bar.

8. The orthosis of claim 6, wherein the support structure comprises a horizontal portion extending proximally.

9. The orthosis of claim 6, wherein the support structure has a lower end attached to the second portion.

10. The orthosis of claim 9, wherein the lower end of the support structure comprises a first bar and a second bar, the first bar being disposed at an angle with respect to the second bar.

11. The orthosis of claim 9, wherein the lower end of the support structure is generally Y-shaped.

12. The orthosis of claim 1, wherein the first portion comprises an anterior projection offset from a longitudinal axis aligned with the at least one joint.

13. The orthosis of claim 1, wherein the at least one joint limits rotation of the first portion with respect to the second portion to approximately twenty degrees in a forward direction and approximately twenty degrees in a rearward direction.

14. The orthosis of claim 1, further comprising an inner liner.

15. The orthosis of claim 1, wherein the ratchet mechanism is a ratchet buckle having a button for releasing all tension when the button is actuated.

16. The orthosis of claim 1, wherein the ratchet mechanism comprises a rotatable handle.

17. The orthosis of claim 16, wherein the handle comprises a rotatable knob.

18. The orthosis of claim 1, wherein at least one of the joint comprises a goniometer.

19. The orthosis of claim 1, wherein the first portion has an anterior projection, the first end of the strap being connected to the anterior projection.

20. The orthosis of claim 1, wherein the at least one joint comprises a lateral joint and a medial joint, each of the lateral joint and the medial joint permitting relative rotation between the first portion and the second portion.
